(19) United States
(12) Patent Application Publication
(10) Pub. No.: US 2011/0196187 A1
(43) Pub. Date: Aug. 11, 2011

(54) MAGNETICALLY SENSITIVE DRUG CARRIERS AND DELIVERY DEVICES

(75) Inventors: Florian Niklas Ludwig, Ebikon (CH); Syed Faiyaz Ahmed Hossainy, Hayward, CA (US); Bjorn Svensson, Gilroy, CA (US); Adam Sharkawy, San Jose, CA (US); Michael J. Cima, Winchester, MA (US); Omid C. Farokhzad, Chestnut Hill, MA (US); Jesus Magana, Redwood City, CA (US); Randolf Von Oepen, Los Altos Hills, CA (US); Mina Chow, Campbell, CA (US); William E. Webler, JR., San Jose, CA (US); Travis R. Yribarren, San Mateo, CA (US); Kevin J. Ehrenreich, San Francisco, CA (US)

(73) Assignee: ABBOTT CARDIOVASCULAR SYSTEMS INC., Santa Clara, CA (US)

(21) Appl. No.: 12/704,092

(22) Filed: Feb. 11, 2010

(51) Int. Cl. A61N 2/00

(52) U.S. Cl. 600/12; 600/9

(57) ABSTRACT

Apparatuses for delivering compositions of matter comprising a magnetically sensitive drug carrier and a related drug and methods for causing the drug carriers to localize within the patient using an internal or external magnetic field are described.

### BACKGROUND

Systemic delivery of drugs to a mammal is many millennia old if one considers medicinal herbs as drugs. When the overall ailment to be treated occurs system wide, systemic delivery is a suitable delivery method. But in some cases of localized diseases, such as vascular or cardiovascular diseases, providing an effective concentration to the treated site using systemic delivery of the medication results in high drug concentrations throughout the patient. These high drug concentrations can produce adverse or toxic side effects. On the other hand, because in local delivery the effective concentration is only high near the local diseased site, local delivery can provide much lower concentrations of medication throughout the rest of the patient. This concentration difference allows local delivery to cause fewer side effects and achieve better results. Unfortunately, local or regional delivery of a drug is much more difficult in many cases. What is needed is a delivery method that allows drug administration in a systemic manner, but also having the capability to act only or predominantly locally in the patient, thus keeping the system-wide drug concentration low while providing an effective concentration within the diseased region or at the diseased site.

A common method of visualizing the human vascular system is through angiography, otherwise known as fluoroscopy. Fluoroscopy involves the introduction of a radiopaque contrast agent within a patient's vascular system that is subsequently imaged using x-ray equipment. By absorbing the x-rays, the contrast appears dark against the surrounding tissue, and a physician can use this distinction to appreciate changes in vascular geometry that indicate diseased vessel narrowing. The technique is widely used and provides the advantage of being well understood and relatively economical as an imaging and diagnostic tool.

More recently, intravascular ultrasound (IVUS) has provided an alternative method of diagnosing plaque deposits within the vascular system and the stenoses that they cause. This technology commonly includes an ultrasound probe connected to a catheter that may be placed within the patient's anatomy in order to relay ultrasonic imaging data to a visual display that allows the physician to understand the tissue constituency and vascular geometry where the physician has positioned the probe. This technique provides the useful advantage of allowing the physician to not only understand the vascular geometry, but also to view the distribution of plaque throughout the vasculature and along the vessel walls. Of course, the technology is also more expensive due to its less widespread use and comparative higher sophistication.

### SUMMARY

In accord with an embodiment of the invention, a method comprising delivering a magnetically sensitive drug carrier near a region of the vasculature and applying magnetic energy to the vasculature is described. In this embodiment or in other embodiments, applying magnetic energy causes a change in motion of the drug carrier and sometimes localizes the drug carrier particles in a particular region.

In these or other embodiments, the delivery is accompanied by intravenous ultrasound imaging of a vascular legion.

In these or other embodiments, applying magnetic energy uses a percutaneous magnetic source apparatus. In some embodiments, this magnetic source apparatus has a magnetic source attached to a distal end. This distal end can be placed into a heart chamber, coronary artery or other vessel before, during, or after, placement of the delivery apparatus into the same or different heart chamber, coronary artery or other vessel. In some embodiments, the distal end of the magnetic source is placed into a heart chamber, coronary artery or other vessel before during or after placement of the delivery apparatus into an adjacent chamber, artery, or vessel.

In these or other embodiments, the magnetic source is a permanent magnet or an electromagnet.

In some embodiments, the distal end connects to an expandable member, which in some embodiments has an outer surface coated or impregnated with a magnetically sensitive drug carrier.

In these or other embodiments, the expandable member is a porous balloon, self-deployable foam, or a self-deployable cage-supported membrane.

### BRIEF DESCRIPTION OF THE FIGURES

FIG. 1 shows a schematic view of an apparatus of the current invention.

FIG. 2 shows a schematic view of a heart showing the placement of a probe into a heart chamber.

FIG. 3 shows a schematic view of a heart showing an alternative placement of a probe into a heart chamber.

FIG. 4 shows an expanded view of a portion of FIG. 3.

FIG. 5 shows cross-section A-A of FIG. 4.

FIG. 6 shows a view of a coronary artery and vein with a magnetic probe and a delivery apparatus.

FIG. 7 shows a view of a delivery apparatus and optional occlusion balloon.

FIG. 8 is a schematic representation of a photograph of a model vessel at time t=0 sec.

FIG. 9 is the same as FIG. 8 except t=60 sec.

FIG. 10 is the same as FIG. 8 except t=140 sec.

FIG. 11 is the same as FIG. 8 except t=280 sec.

### DETAILED DESCRIPTION

Embodiments of this invention use a magnetically sensitive drug carrier that is simple to administer from a catheter or other percutaneous delivery apparatus during an angiogram, angioplasty, or the like. In some embodiments, drug-loaded, biocompatible ferromagnetic nanoparticles serve as a magnetically sensitive drug carrier; although, other formulations are useful, as well.

The following description of several embodiments describes non-limiting examples that further illustrate the invention. All titles of sections contained in this document, including those appearing above, are not invention limitations, but rather serve to provide structure to the illustrative description of the invention that is provided by the specification.

Unless defined otherwise, all technical and scientific terms used in this document have the meanings that those skilled in the art of the invention commonly understand them to have. The singular forms “a”, “an”, and “the” encompass plural forms unless the context clearly indicates otherwise.

While the description speaks in terms of a magnetically sensitive drug carrier, a drug, therapeutic substance, or bioactive agent molecule or agglomeration that itself has magnetic sensitivity, as described below, would fall within the scope of this description and claims. That is, such a molecule is defined as a magnetically sensitive drug carrier for purposes of this document.

This invention discloses the use of magnetically sensitive drug carriers with a magnetic field to target therapeutic agents to the carotid arteries, coronary arteries, or other desired treatment region. Drug-loaded, magnetically sensitive carriers are delivered systemically. The delivery described in this document avoids the problems typically associated with systemic delivery by localizing the particles in or near the desired treatment region. To localize and retain these particles in the vasculature, such as in heart chambers, coronary arteries, other vessels, or other desired treatment regions, a magnetic field is applied using any number of methods as discussed below. Once administered, these drug carriers release the drug over a preselected time at their localized site.

Various embodiments of this invention provide a mechanism for efficient drug delivery to the arterial tree. For instance, a drug carrier is formulated to be responsive to an induced magnetic field. This formulation thus becomes a magnetically sensitive drug carrier and is applied to a biological system using systemic administration or local or regional administration, for example, to the pericardial sac or some other location accessible from the patient's vasculature. A magnetic field created by a device, for example, an intravascular catheter with a magnet, will attract the drug to the site to promote arterial loading of the drug. This device might also be a permanent implant, such as an implanted magnet, or created from an external source, such as an external magnetic field. The field could be a fluctuating field to enhance penetration of the particles. This method would allow the physician to guide the magnetically sensitive drug carrier to the target site or region, and further may allow increased or controlled arterial concentrations, region-specific delivery, or time-controlled delivery of the magnetically sensitive drug carrier. The drug would then be released from the formulation to influence a desired biological process. The magnetic field may be induced rapidly after administration of the magnetically sensitive drug carrier, or may occur later, or at multiple times.

### Magnetically Sensitive Drug Carrier

In various embodiments, magnetically sensitive drug carriers can be nanoparticles or microparticles, liposomes, micelles, nano-fibers, hydrogels, or the like. The magnetic sensitivity can reside in the base material of the particle or a separate material with magnetic sensitivity can be added to the particle during or after the particle's manufacture. Depending upon the delivery method, the particles of the magnetically sensitive drug carriers can range in size from 1 nanometer for ferrous compound particles to several microns for some liposomes.

### Magnetic Particles and Beads

Magnetic particles and beads sourced from or made similarly to beads sourced from the companies listed below are useful in the practice of the current invention.
Sera-Mag™ magnetic particles are based on U.S. Pat. No. 5,648,124, and use 1 μM magnetic carboxylate-modified base particles made by a core-shell process. The entire contents of U.S. Pat. No. 5,648,124 are hereby incorporated by this reference;
Iron-containing nanoparticles available from Ocean Nanotech;
Functionalized magnetic beads such as those available from Bioclone, Inc.;
Coated magnetic particles such as those available from Spherotech; and
Coated magnetic beads such as those available from ThermoScientific.

### Liposomes

Liposomes available from Encapsula Nano Sciences are also useful as the magnetically sensitive drug carrier. Other liposomes useful in the practice of this invention can be made by methods disclosed in the following references:
Bimodal Paramagnetic and Fluorescent Liposomes for Cellular and Tumor Magnetic Resonance Imaging; Kamaly, Nazila; Kalber, Tammy; Ahmad, Ayesha; Oliver, Morag H.; So, Po-Wah; Herlihy, Amy H.; Bell, Jimmy D.; Jorgensen, Michael R.; Miller, Andrew D.; Imperial College Genetic Therapies Centre, Department of Chemistry, Imperial College London, London, UK; Bioconjugate Chemistry (2008), 19(1), 118-129;
Preparation and characterization of novel magnetic cationic polymeric liposomes. Liang, Xiao-Fei; Wang, Han-Jie; Tian, Hui; Luo, Hao; Cheng, Jing; Hao, Li-Juan; Chang, Jin. Institute of Nanobiotechnology, School of Materials Science and Engineering, Tianjin University, Tianjin, Peop. Rep. China. Gaodeng Xuexiao Huaxue Xuebao (2008), 29(4), 858-861;
The effect of magnetic targeting on the uptake of magnetic-fluid-loaded liposomes by human prostatic adenocarcinoma cells. Martina, Marie-Sophie; Wilhelm, Claire; Lesieur, Sylviane. Equipe Physico-Chimie des Systemes Polyphases, CNRS UMR 8612, Chatenay, Malabry, Fr. Biomaterials (2008), 29(30), 4137-4145;
Preparation and use of magnetically guided liposomes in treatment of oncological diseases. Alyautdin, R. N.; Torshina, N. L.; Cherkasova, O. G.; Filippov, V. I.; Larin, M. Yu.; Ivanov, P. K.; Blokhin, D. Yu.; Bayburtskiy, F. S. I. M. Sechenov Medical Academy, Moscow, Russia. Oxidation Communications (2006), 29(4), 924-931;
Biotechnology of Magnet-Driven Liposome Preparations. Ismailova, G. K.; Efremenko, V. I.; Kuregyan, A. G. State Pharmaceutical Academy, Pyatigorsk, Russia. Pharmaceutical Chemistry Journal (2005), 39(7), 385-387; and
Injectable magnetic liposomes as a novel carrier of recombinant human BMP-2 for bone formation in a rat bone-defect model. Matsuo, Toshihiro; Sugita, Takashi; Kubo, Tadahiko; Yasunaga, Yuji; Ochi, Mitsuo; Murakami, Teruo. Department of Orthopaedic Surgery, Programs for Applied Biomedicine, Division of Clinical Medical Science, Graduate School of Biomedical Sciences, Hiroshima University, Hiroshima, Japan. Journal of Biomedical Materials Research, Part A (2003), 66A(4), 747-754.

### Micelles

Micelles useful in the practice of this invention can be made by methods disclosed in the following references:
Synthesis and surface engineering of superparamagnetic iron oxide nanoparticles for drug delivery and cellular targeting. Gupta, Ajay Kumar; Gupta, Mona. Formulation Development Department, Torrent Research Centre, Torrent Pharmaceutical Limited, Gujarat, India. Editor(s): Kumar, M. N. V. Ravi. Handbook of Particulate Drug Delivery (2008), 1 205-221.
Micellar hybrid nanoparticles for simultaneous magnetofluorescent imaging and drug delivery. Park, Ji-Ho; von Maltzahn, Geoffrey; Ruoslahti, Erkki; Bhatia, Sangeeta N.; Sailor, Michael J. Materials Science adn Engineering Program, Department of Chemistry and Biochemistry, University of California, San Diego, La Jolla, Calif., USA. Angewandte Chemie, International Edition (2008), 47(38), 7284-7288.
Preparation and characterization of PNIPAAm-b-PLA/Fe₃O₄ thermo-responsive and magnetic composite micelles. Ren, Jie; Jia, Menghong; Ren, Tianbin; Yuan, Weizhong; Tan, Qinggang. Institute of Nano and Bio-Polymeric Materials, School of Material Science and Engineering, Tongji University, Shanghai, Peop. Rep. China. Materials Letters (2008), 62(29), 4425-4427.
cRGD-encoded, MRI-visible polymeric micelles for tumor-targeted drug delivery. Gao, Jinming; Nasongkla, Norased; Khemtong, Chalermchai. Simmons Comprehensive Cancer Center, University of Texas Southwestern Medical Center, Dallas, Tex., USA. Editor(s): Amiji, Mansoor M. Nanotechnology for Cancer Therapy (2007), 465-475;
Diacyllipid Micelle-Based Nanocarrier for Magnetically Guided Delivery of Drugs in Photodynamic Therapy. Cinteza, Ludmila O.; Ohulchanskyy, Tymish Y.; Sahoo, Yudhisthira; Bergey, Earl J.; Pandey, Ravindra K.; Prasad, Paras N. Institute for Lasers Photonics and Biophotonics, SUNY at Buffalo, Buffalo, N.Y., USA. Molecular Pharmaceutics (2006), 3(4), 415-423;
Synthesis of magnetic nanoparticles and their application to bioassays. Osaka, Tetsuya; Matsunaga, Tadashi; Nakanishi, Takuya; Arakaki, Atsushi; Niwa, Daisuke; Iida, Hironori. Department of Applied Chemistry, Waseda University, 3-4-1 Okubo, Shinjuku, Japan. Analytical and Bioanalytical Chemistry (2006), 384(3), 593-600;
Magnetite-loaded polymeric micelles as ultrasensitive magnetic-resonance probes. Ai, Hua; Flask, Christopher; Weinberg, Brent; Shuai, Xintao; Pagel, Marty D.; Farrell, David; Duerk, Jeffrey; Gao, Jinming. Department of Biomedical Engineering Case Western, Reserve University, Cleveland, Ohio, USA. Advanced Materials (Weinheim, Germany) (2005), 17(16), 1949-1952; and
Reverse Micelle Synthesis and Characterization of Superparamagnetic MnFe₂O₄ Spinel Ferrite Nanocrystallites. Liu, Chao; Zou, Bingsuo; Rondinone, Adam J.; Zhang, Z. John. School of Chemistry & Biochemistry, Georgia Institute of Technology, Atlanta, Ga., USA. Journal of Physical Chemistry B (2000), 104(6), 1141-1145.

### Hydrogel

Hydrogels useful in the practice of this invention can be made by methods disclosed in the following references:
Study on controlled drug permeation of magnetic-sensitive ferrogels: Effect of Fe₃O₄ and PVA. Liu, Ting-Yu; Hu, Shang-Hsiu; Liu, Kun-Ho; Liu, Dean-Mo; Chen, San-Yuan. Department of Materials Sciences and Engineering, National Chiao Tung University, Hsinchu, Taiwan. Journal of Controlled Release (2008), 126(3), 228-236;
Controlled Pulsatile Drug Release from a Ferrogel by a High-Frequency Magnetic Field. Hu, Shang-Hsiu; Liu, Ting-Yu; Liu, Dean-Mo; Chen, San-Yuan. Department of Materials Sciences and Engineering, National Chiao Tung University, Hsinchu, Taiwan. Macromolecules (Washington, D.C., United States) (2007), 40(19), 6786-6788;
Composites of polymeric gels and magnetic nanoparticles: preparation and drug release behavior. Francois, Nora J.; Allo, Sabina; Jacobo, Silvia E.; Daraio, Marta E. Laboratorio de Aplicaciones de Polimeros Hidrofilicos, Departamento de Quimica, Facultad de Ingenieria, Universidad de Buenos Aires, Buenos Aires, Argent. Journal of Applied Polymer Science (2007), 105(2), 647-655;
Synthesis and temperature response analysis of magnetic-hydrogel nanocomposites. Frimpong, Reynolds A.; Fraser, Stew; Hilt, J. Zach. Department of Chemical and Materials Engineering, University of Kentucky, Lexington, Ky., USA. Journal of Biomedical Materials Research, Part A (2006), Volume Date 2007, 80A(1), 1-6;
PVP magnetic nanospheres: Biocompatibility, in vitro and in vivo bleomycin release. Ding, Guowei; Adriane, Kamulegeya; Chen, XingZai; Chen, Jie; Liu, Yinfeng. Tongji university hospital, Shanghai, Peop. Rep. China. International Journal of Pharmaceutics (2007), 328(1), 78-85; and
Preparation and characterization of magnetic targeted drug controlled-release hydrogel microspheres. Chen, Jie; Yang, Liming; Liu, Yinfeng; Ding, Guowei; Pei, Yong; Li, Jian; Hua, Guofei; Huang, Jian. Department of Chemical Engineering and Technology, Shanghai University, Shanghai, Peop. Rep. China. Macromolecular Symposia (2005), 225(Polymers in Novel Applications), 71-80.

### Polymeric Nanoparticles

Polymeric Nanoparticles useful in the practice of this invention can be made by methods disclosed in the following references:
Seung-Jun Lee et al. Journal of Magnetism and Magnetic Materials 272-276 (2004) 2432-2433;
S. A. Gomez-Lopera et al. Journal of Colloid and Interface Science 240,40-47 (2001);
L. Ngaboni Okassa et al. International Journal of Pharmaceutics 302 (2005) 187-196;
Seung-Jun Lee et al. Colloids and Surfaces A: Physicochem. Eng. Aspects 255(2005) 19-25; and
Bryan R. Smith et al. Biomed Microdevice (2007) 9:719-727.

Drug-loaded particles comprise a magnetically sensitive component such as ferrite particles, ferrous oxide (iron oxide), rare earth particles, and the like. Particles may comprise polymer, degradable polymer, biodegradable glass or biodegradable metal, lipids, and the like.

In some embodiments, the magnetic agents are encapsulated into the nanoparticles or other carriers during the encapsulation process (e.g. emulsion, spray drying, and electrospraying, etc.) without interacting with the drugs or destroying the magnetic character of the magnetic agent.

In some embodiments, the magnetically sensitive drug carrier may comprise an oxidizing agent. The particle size for some embodiments of this magnetically sensitive drug carrier would be <I micron, and preferably <500 nm, to increase the ability of the particle to migrate through the tissue. This particle would be delivered into the pericardial sac with the use of a surgical technique, or using an intravascular approach, delivered to create a reservoir of a magnetically sensitive drug carrier comprising, as the drug component, an antioxidant. Subsequently, at a desired time, such as following a myocardial infarction, a catheter could be introduced into the coronary tree, and positioned in a region of affected ischemic tissue, near the infarction site. A magnetic field generated from this device would draw the particles to the arterial site. At this arterial site, the particles would deliver the antioxidant to influence infarct progression.

In some embodiments, the magnetically sensitive drug carrier comprises a therapeutic agent that may be functionalized in the manufacturing process by adding a magnetic or paramagnetic material to the agent mixture. For example, iron particles may be coated with the therapeutic agent, and those particles may subsequently be impregnated within the surface of the expandable structure of an percutaneous delivery apparatus, as discussed below. The iron particles may be magnetized to increase the forces between the particles in the magnetic source, which improves the uptake of the desired therapeutic agent, in some embodiments. Alternatively, it may be possible to charge the therapeutic agent ionically in order to further functionalize it in accordance with this invention.

### Methods of Associating Magnetically Sensitive Drug Carrier with the Drug or Drugs

The drug or drugs can be attached to or contained in the magnetically sensitive carrier in a variety of ways. In various embodiments, the drug is within the particle (internal to the particle), located within pores in the particle (for porous particles), adsorbed on the surface of the particle, conjugated to the surface of the particle, or simply mixed with the particle material.

In some embodiments, the magnetic nanoparticles (such as metal oxide particles) conjugate with the therapeutic agents through a cleavable linker. The linker's design allows it to release the drug component by acid hydrolysis, reduction, oxidation, or photochemical or enzymatic action either present in the tissue or induced externally. The linker is an assembly of atoms attached to one another, in some embodiments, through chemical bonds. The linker, in some embodiments, attaches to at least two pieces: the drug moiety and the magnetically sensitive carrier moiety. In some embodiments, the attachment occurs through chemical bonds—sometimes covalent bonds.

Cells and other biological carriers that have been pretreated to contain internal magnetic nanoparticles may be injected into a patient's circulatory system and then be attracted to a specific target by placing an internal or an external magnetic field at a desired target site once the cells are circulating.

### Drug Transfer from Particle to Tissue

Once the magnetically sensitive drug carrier has been localized by applying the magnetic field, the drug should leave the particle and enter the tissue or diseased tissue at the treatment site. For particles in which the drug is absorbed into or adsorbed onto the particle, this “leaving” most likely is influenced by diffusion. In some embodiments, diffusion may be the rate-limiting step. For particles in which the drug is absorbed into pores in the particle, this “leaving” most likely is influenced by diffusion out of the pores. For particles in which the drug is attached such as through a bond directly to the drug or through a set of linking atoms, this “leaving” most likely is influenced by breaking the bond between the drug and the particle. In some embodiments, the rate-limiting step, after localizing the magnetically sensitive particles, in the process of the drug moving from a particle to the tissue, is breaking the bond or bonds between the particles and the drug. In some embodiments, the drug may be able to act on the tissue without “leaving” the particle.

The magnetic field will direct the conjugated drugs to the target site where the drug will release from the formulation over time.

Various embodiments of this invention are useful for the treatment of vascular dysfunction in which local delivery of a drug, in a controlled or reoccurring manner, would be beneficial, such as chronic arterial disease. This invention is used for treating any locally manifesting disease in which controlled dosing of a drug at a specific location would be beneficial.

Additionally, this invention may also be used to treat other vessels or tissue, including cancer located close to the surface or otherwise having appropriate vascular access.

### Generation of Magnetic Field

The magnetically sensitive drug carrier will be attracted to the delivery site with a magnetic field created by a device, for example, by an intravascular catheter device with a ferromagnet, to promote arterial loading of the drug. This device may also be a permanent implant, such as an implanted magnet (including a magnet located in or on a bare-metal or drug-eluting stent), or an external magnet or magnetic field. The field may be a fluctuating field to enhance penetration of the particles.

For purposes of this document, magnetic field means (1) a magnetic field with its accompanying field gradient caused by the natural decrease in field strength as the distance to the source of the magnetic material increases; (2) an engineered magnetic field gradient that is purposely constructed, such as with an electromagnetic solenoid or a permanent or electromagnet with poles shaped to provide the desired gradient; or (3) a combination of (1) and (2).

The magnetic fields can be from one or more electromagnets or permanent magnets. These magnets can be outside the patient, inside the patient, or a combination of both. External fields have the advantage of being easier and more convenient to apply to the patient. On the other hand, since magnetic field strength diminishes rapidly as the distance from the magnet to the target increases, external magnetic field sources need to be much more intense than internal magnetic field sources. In addition to distance, the shape of the magnet greatly affects the resulting field. The shape dependency allows tailoring the shape to provide a field suitable for desired particle localization method. For instance, properly shaped electromagnets or permanent magnets could cause a large magnetic field or large magnetic field gradient to center on the area to be treated, such as the heart or cardiovascular system. Similarly, using an electromagnet, the magnetic field can be turned on and off or otherwise pulsed, for instance between two different field strengths. (This would help the particle to penetrate the tissue or embed in the tissue better).

Magnetic stereotaxis systems exist and are currently used to steer catheter tips in complex vascular anatomy using external magnetic fields. See J Neurosurg. 2000 August; 93(2):282-8. The hardware is available from Stereotaxis Corporation.

Magnetic carriers can be used not only for local therapy but also for “regional therapy” by varying the intensity of magnetic field along the target region. As a result drug loading and delivery can be controlled with the variation of the externally applied field.

Magnetic carriers can be released from a specific, magnetically induced repository to the systemic circulation over time by adjusting the time decay of magnetization of these particles to the desired release rates.

In addition to the above, magnetization decay and thus release rates can be further controlled via energy modalities such as heat.

### Magnetic Materials

A magnetically sensitive drug carrier requires enough magnetic material to be sensitive to or to respond to a magnetic field. For purposes of this document, respond means that the magnetic field is capable of causing a change in the motion of the magnetically sensitive drug carrier particles. Thus, one of ordinary skill in the art appreciates that enough magnetic material depends, in part, on the size of the particle, the magnitude or shape of the magnetic field, the distance to the magnetic field, or the magnetic strength of the magnetic material (otherwise known as the magnetization M).

In some embodiments, respond to the magnetic field means that the drug carrier experiences a change in motion (due to the magnetic field) such that drug delivery is improved in any way over the same drug carrier absent the magnetic field source. In some embodiments, respond to the magnetic field means that the particles are directed to the desired treatment area long enough to improve or increase the drug transfer from the drug carrier to the target tissues versus the drug carrier in the absence of the magnetic field.

In some embodiments, respond to the magnetic field means that the drug carrier experiences a change in motion (due to the magnetic field) such that drug delivery is improved in any way over the same drug carrier absent the magnetic field source. In some embodiments, response of magnetic field means that the particles are directed to the desired treatment area long enough to improve or increase the drug transfer from the drug carrier to the tissue versus the drug carrier in the absence of the magnetic field. Beneficial changes in any of the following parameters can be used as indices of efficacy. In some cases, parameter classes include those related to tissue composition, such as lipid composition, to inflammation, to apoptosis, to fibrosis etc. Alternatively or additionally, parameter classes include those related to function such as changes in blood flow, oxygenation, electrophysiology etc.

There are other ways to characterize the response to the magnetic field, as well. Usually, the amount (concentration) of drug in the target tissues has units like, nanograms of drug per gram of tissue. There is usually a minimum effective dose of the drug in question. Thus, in some embodiments, to respond to the magnetic field means that because of the magnetic field the particles stay within the desired treatment area long enough to allow drug transfer. The drug transfer is significant enough that the concentration of the drug in the target tissues rises above the minimum effective dose to be therapeutically significant. Alternatively, to respond to the magnetic field means that because of the magnetic field the particles stay within the desired treatment area long enough to allow drug transfer significant enough that the time that the concentration of the drug in the target tissue is above the minimum effective dose is therapeutically significant. Therapeutically significant usually means that the therapy provides a detectable improvement in an objective measurement of a disease parameter (like restenosis rate, vessel ID, ejection fraction, etc.) or a detectable slowing of progression in the disease symptoms (like angina, walking distance, CHF class) or lowered death rates.

Ferromagnetic materials are useful magnetic materials in the magnetically sensitive drug carrier. These materials have permanent magnetic moments, hence magnetism on a macroscopic scale. Ferromagnetic materials have magnetic domains that each have a magnetic moment simplistically made up of the contributions of the unpaired electrons on the atoms (or in some cases, molecules) of the material. In the absence of thermal energy in the ferromagnetic material, all of the magnetic moments of the magnetic domains would align. But at room temperature, for instance, the thermal energy causes misalignment between the magnetic moments of the domains. Nonetheless, at least some residual alignment remains yielding magnetism in the material.

Thus, ferromagnetic materials are useful for inclusion in the magnetically sensitive drug carriers described in this document, if they have the other chemical properties necessary to be safe for use in pharmaceutical compositions. Ordinarily skilled artisans know these properties well.

Moreover, paramagnetic and super-paramagnetic materials could be used as the magnetic material for the magnetically sensitive drug carriers described in this document. Since these materials do not have a permanent magnetic moment at treatment temperatures, their use as a magnetic component of the magnetically sensitive drug carrier requires two magnetic fields or at least one field gradient. One of these magnetic fields causes the magnetic moments in the materials to align, giving them the ability to respond to a magnetic field; the other magnetic field causes the localization (as this term is used in the current disclosure) of the aligned paramagnetic atoms or molecules contained in the magnetically sensitive drug carriers. Examples of suitable paramagnetic materials include iron oxide, platinum, and tungsten.

The force exerted on magnetically responsive particles is proportional to the gradient of the magnetic field and the magnetic moment of the particle. In cases where the magnetic moment is induced, e.g. in the case of paramagnetic or superparamagnetic particles, the particle magnetic moment, and therefore the force exerted on it, becomes also a function of the magnitude of the external magnetic field.

Specific compositions of useful magnetically sensitive components of the magnetically sensitive drug particles include certain elements and compounds. Elements can be paramagnetic if they have unpaired electrons. The following are some examples of paramagnetic elements:
Aluminum (metal)
Barium (metal)
Oxygen (non-metal)
Platinum (metal)
Sodium (metal)
Strontium (metal)
Uranium (metal)
Technetium (metal)
Dysprosium (metal)—ferromagnetic

Many salts or compounds of the d and f transitional metal groups exhibit paramagnetic behavior. The following are some examples of paramagnetic compounds:
Copper sulphate
Dysprosium oxide
Ferric chloride
Ferric oxide
Holmium oxide
Manganese chloride

### Intravascular Ultrasound

Nearly all atherosclerotic lesions are eccentric. The orientation and location of the thickest, most diseased, region of the lesion can be identified by IVUS. Administration of magnetic drug delivery microspheres or nanoparticles can be made via catheter to the site. This can be done combined with a properly oriented external magnetic field, which will attract the particles towards, and possibly into, the thicker part of the lesion thereby directing the particles towards a more diseased region of the lesion.

### Percutaneous Apparatus

Regional therapy of the vascular system can be achieved by delivery of a therapeutic agent into the vessel wall. This delivery can occur through a number of delivery routes and modes based on their ability to allow entry of an effective amount of substance. It is possible to deliver the therapeutic agent endoluminally without injuring the vessel wall. Such delivery could be a preferred method if it permits an effective amount of substance to enter and remain within the vessel wall and if it meets other therapeutic criteria. For example, the treatment method should allow a vessel length of about 2-3 cm to be treated during an intervention, and it may permit delivery of particles in the 10 nm to 20 micrometer range. Embodiments of the invention that are described in this document meet these criteria by promoting delivery of therapeutic agent into the arterial wall. In general, embodiments of the invention use magnetic forces to attract particles to the vessel wall promote adhesion with the luminal surface of the vessel wall. This is necessary for the vessel wall to take up a particle agent, which over time will migrate through the endothelial cell (EC) and internal elastic lamina (IEL) layers into the vessel wall.

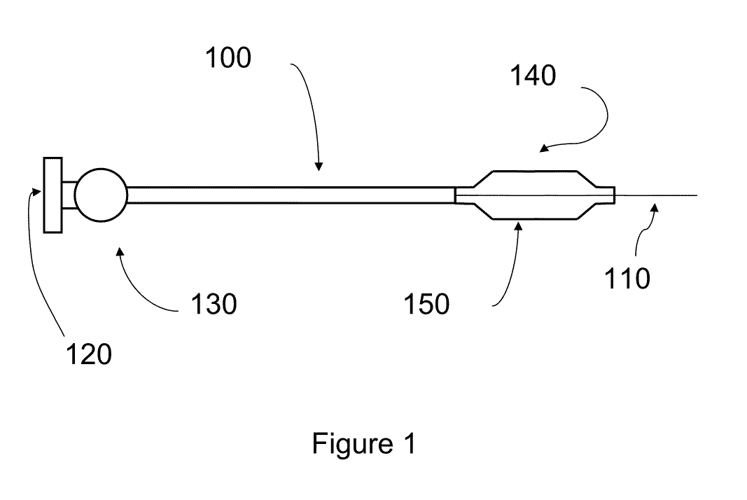

In one embodiment of the invention, as shown in FIG. 1, the invention includes an elongated catheter 100 that can include a guidewire lumen 120 with a guidewire 110 through its length. This can guide the catheter 100 through the vascular system from an entry site such as femoral artery to the treatment site such as a location of vulnerable plaque within a coronary vessel. The catheter 100 may therefore include a proximal end 130 for moving the catheter 100, and a distal working end 140 that may include an expandable member 150 such as a balloon (shown expanded in FIG. 1). Other expandable members may replace the balloon in accordance with the present invention. For example, the expandable member 150 may be an expandable nitinol cage or an expandable foam cylinder. In some embodiments, the expandable member 150 may have a therapeutic-agent-coated or -impregnated outer surface.

The catheter 100 may include needles and, in some embodiments, these needles face toward the vessel wall. These needles may allow the therapeutic agent to flow from the catheter 100 to the vessel wall.

To increase migration and adhesion efficiency further, the expandable structure 150 may comprise a porous balloon, self-deployable foam, or a self-deployable, cage-supported membrane that exposes or confines to some degree the drug agent near or at the arterial vessel wall. The catheter 100 may have a structure such that the expandable structure either does not contact the vessel wall, or only gently contacts the vessel wall, to minimize vessel-wall damage. This is useful in cases where the targeted infusion site contains a vulnerable plaque with thin caps. One benefit of using the catheter 100 to infuse magnetic particles near the vessel wall comes from the shorter distance the particles must travel between the exit from the catheter 100 and the vessel wall. Another is that infusion from the catheter 100 increases the number of particles at the vessel wall over other delivery methods.

Another component of the system encompassed by embodiments of this invention is a magnetic source for acting on the magnetized material. This magnetic source may have several embodiments as will be described below.

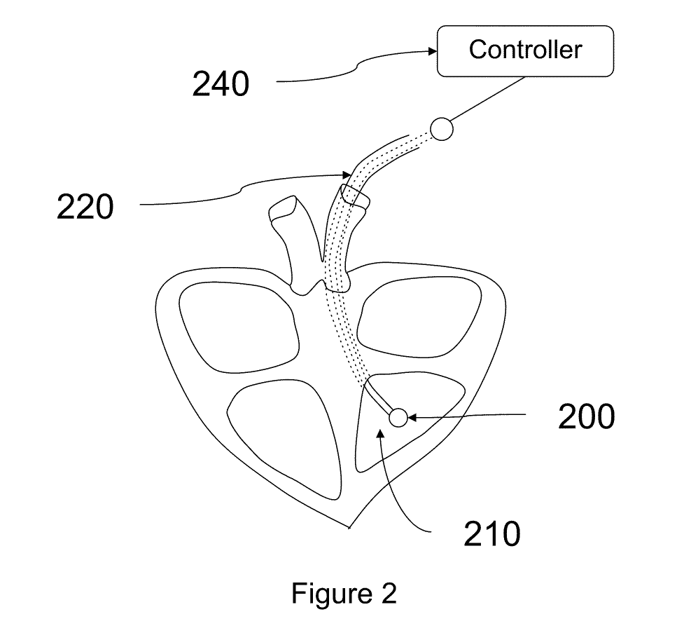

In an exemplary embodiment as illustrated in FIG. 2, a magnetic probe 200 is placed into a chamber of the heart (e.g. ventricle 210). The probe 200 serves to provide magnetic energy to the system. This probe 200 may be incorporated within a catheter 220. The probe may be an inherently magnetic or magnetized material or an electromagnet (operated by a controller 240).

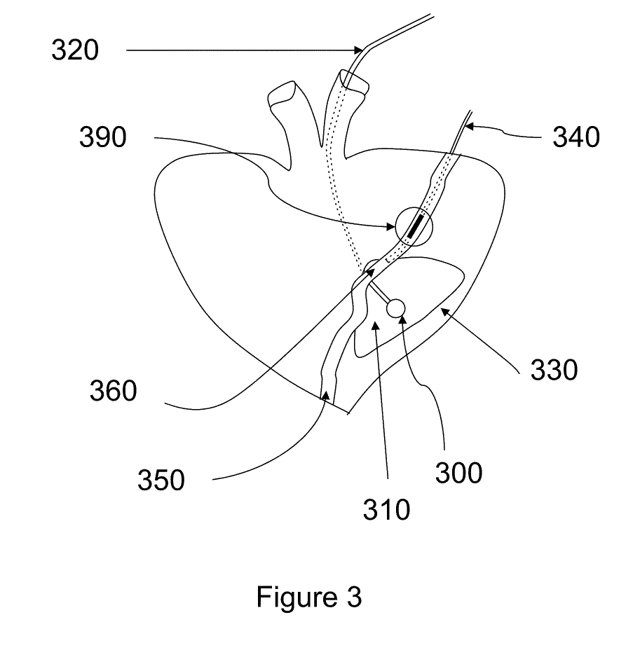

In FIG. 3, a magnetic probe 300 placed in the ventricle 310 (either the left or the right, depending on whether the drug will be infused into the RCA or LAD/LCX) may be housed within the distal segment of a catheter 320. The catheter 320 may be pre-shaped or be a deflectable or steerable catheter 320 such that the distal segment can be brought close to the myocardial wall 330 near the target arterial segment 390 (RCA or LAD/LCX) for the therapy. This will help to increase the magnetic attraction force to the drug-loaded magnetic particles flowing through that targeted segment of the artery. The drug is then infused into the target arterial segment 390 with an arterial catheter 340.

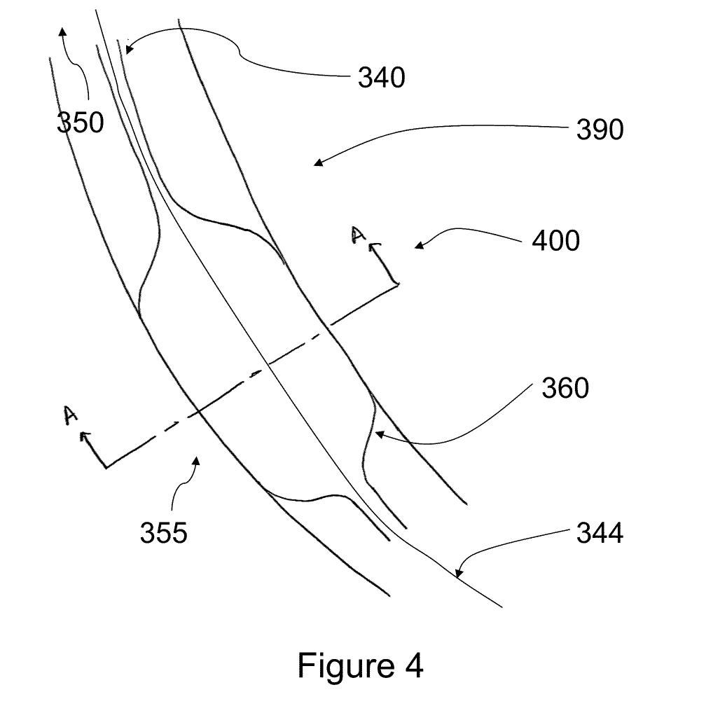

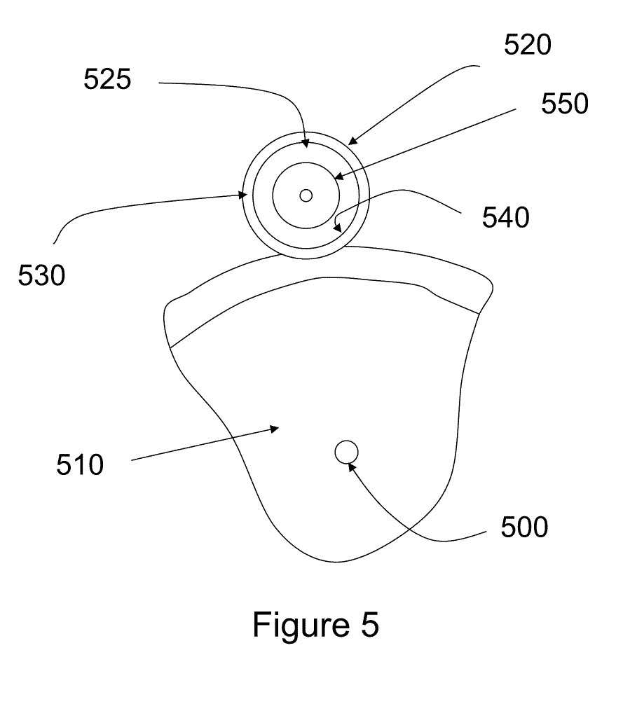

FIG. 4 shows an exploded view of the target arterial segment 390 of FIG. 3, with an A-A cross-section indicated at 400 and depicted in FIG. 5. The arterial catheter 340 may include a guidewire 344 through its length. The arterial catheter 340 may include a proximal end 350 that can be used to move the catheter 340. Distal working end 355 may include an expandable member 360 such as a balloon.

For example, as depicted in FIG. 5, if a coronary artery 520 is to be treated, the magnetic probe 500 may be positioned in the adjacent heart chamber 510 and the particles applied in the artery 520, as previously described. In such a case, the drug will be delivered through a catheter 550, for example, to the arterial lumen 525 adjacent to the chamber 510 with the magnetic probe 500. The magnetic probe 500 in the chamber 510 helps to attract magnetically sensitive drug particles to adhere to the luminal wall 540 of the artery 520 and allow subsequent uptake into the arterial wall 530.

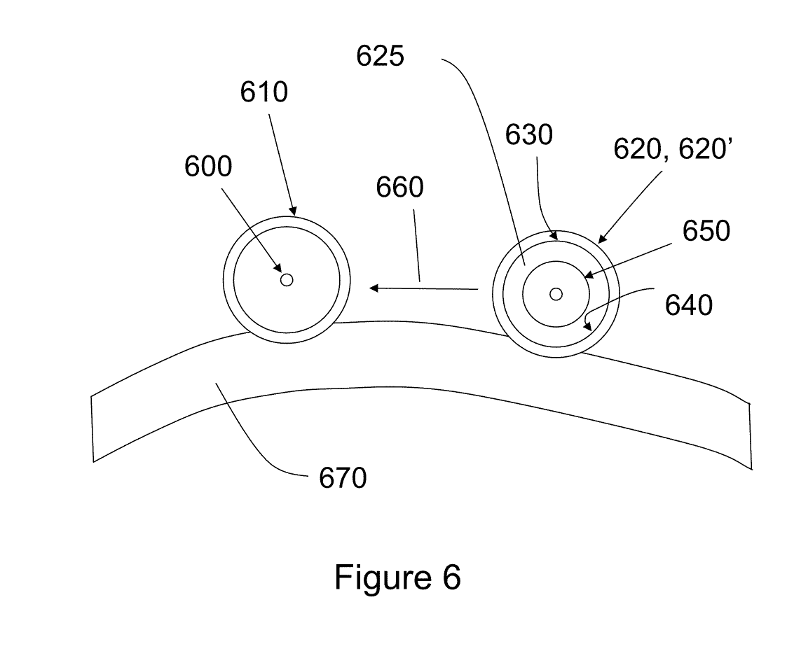

Alternatively, as shown in FIG. 6, the magnetic probe 600 may be incorporated within an elongated catheter sized to be positioned in a vessel adjacent 610 to the treatment vessel 620. This embodiment provides the advantage of acting on the magnetic material from an even closer range, improving uptake. For example, if a coronary artery 620′ is to be treated, the magnetic probe 600 may be positioned in an adjacent vein 610 and the particles applied in the artery 620′, as previously described. The coronary artery 620′ and the adjacent vein 610 are shown on top of the myocardium 670. In this case, the drug will be delivered through a catheter 650, for example, to the arterial lumen 625 adjacent to the vein 610 with the magnetic probe 600. The magnetic probe 600 in the vein 610 helps to attract drug agent particles to adhere to the luminal wall 640 of the artery 620′ and allow subsequent uptake into the arterial wall 630 in the direction shown by arrow 660.

In another embodiment, a magnetic source may be applied extracorporeally. In this embodiment, the magnetic source may either be a permanent ferrous or rare earth magnet or some other type of magnet, configured such as in a patch or blanket placed on the patient's chest, or it may be an electromagnet that is energized to provide the magnetic force upon the magnetized material. In another embodiment, the magnetic field of an MRI machine could be used to provide and adjust the magnetic field, possibly as a result of an MRI imaging or diagnosis of a plaque or vulnerable plaque and to guide the catheter source of the particles to the diagnosed plaque.

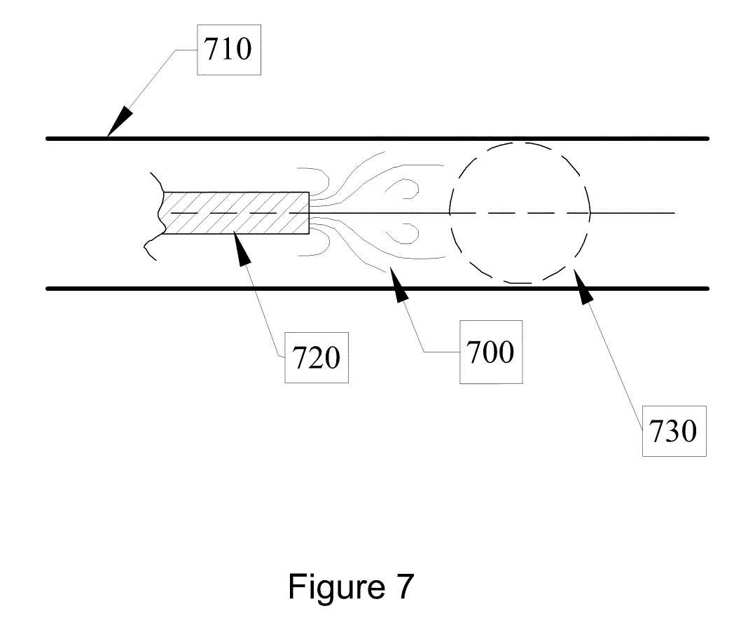

In an alternative embodiment, as shown in FIG. 7, therapeutic agent 700 can be initially placed into the artery 710 by flushing the artery 710 with a solution. This can be achieved by using a catheter 720 such as a diagnostic catheter or other elongated tube. The catheter 720 can be guided to the treatment site, and therapeutic agent 700 can be delivered through the catheter 720. It is also possible to employ a distal occlusion balloon 730 before flushing the treatment site with therapeutic agent 700 in order to prevent washing of the agent 700 away from the desired treatment area.

Once the agent is inserted at the treatment site, the magnetic field may be applied as described previously in order to promote the delivery of the drug into the vessel wall. To promote further particle uptake within the vessel wall, it is possible to either reverse the flow within the target vessel or lower the vessel pressure. In an alternative embodiment, it may be possible to inject magnetic materials into the pericardial sac that surrounds the heart. This material would provide the magnetic field required to act upon the magnetized therapeutic agent solution, in order to draw it into the vessel wall. Delivery of the therapeutic agent into the coronary vessel would occur as described above. But rather than relying solely on the magnetic sources described earlier, the magnetic material within the pericardial sac would force migration of the therapeutic agent into the vessel wall over time.

Methods for treating the coronary vessel is provided by the invention. These will be described with respect to the first magnetic source embodiment, as shown in FIG. 3. That is, the magnetic probe 300 is placed in the ventricular chamber 310. Although, one will appreciate that a similar method may be used with any of the magnetic source embodiments, as described above, with similar effect. First, the delivery catheter 340 is tracked through the vascular system 350 until the distal end 360 is located near the treatment site 390. In the case of a protective sheath placed over the drug-loaded section, the sheath is then retracted to expose the loaded section such that the therapeutic agents can disperse from the catheter when energized. Next, the magnetic probe 300 is tracked into the ventricular chamber 310 and positioned as close to the distal end 360 of the delivery catheter 340 as possible. The probe 300 is then energized, by activating the magnetic source, which causes a magnetic force to be applied to the magnetized particles. The particles are displaced and drawn into the coronary vessel wall, where the therapeutic agent disperses and treats the vascular disease.

The methods employed during the use of the other device embodiments are substantially similar to this method. They differ mainly in the location of the magnetic source, the type of magnetic source used, and the fact that in the arterially placed magnetic source, the particles are diamagnetic and repelled rather than attracted by the magnetic probe.

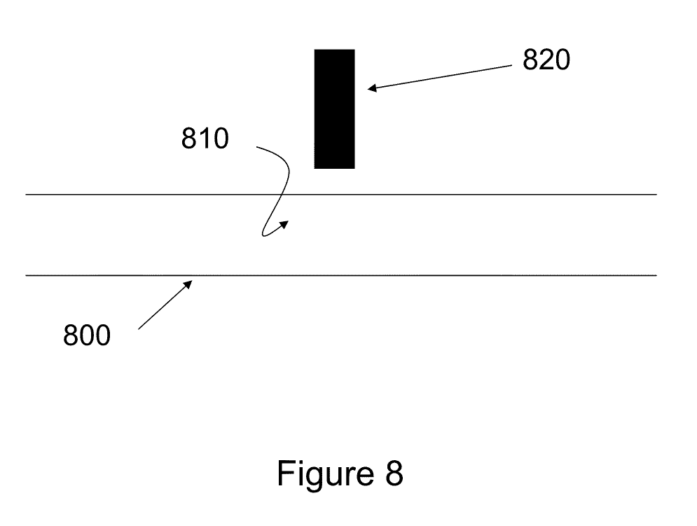

FIG. 8 shows a schematic representation of photographs taken during the experiment described in example 1, below. A model vessel 800 is shown with a magnetic source 820. Magnetic source 820 is arranged near a model desired treatment area 810. FIG. 8 represents time (t) equal to zero. That is, FIG. 8 represents a photograph of the model vessel 800 at the time that magnetically sensitive particles are introduced into the model vessel 800 somewhere upstream of the desired treatment area 810.

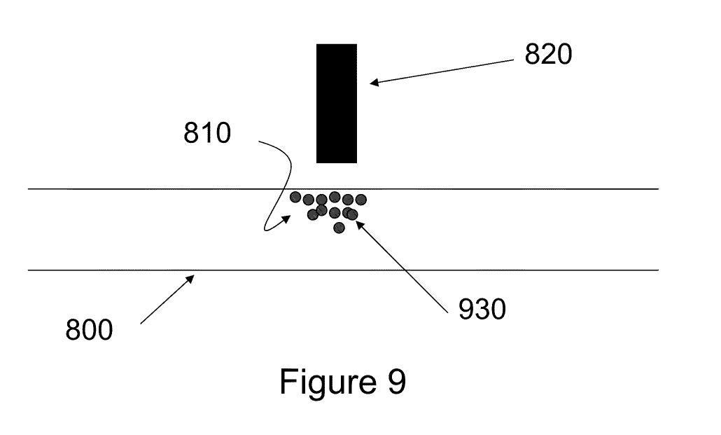

FIG. 9 represents the system at t equal to 60 seconds. That is, FIG. 9 represents a photograph of the model vessel 800 after 60 seconds have passed since the introduction of magnetically sensitive particles 930 into the model vessel 800. In this case, magnetically sensitive particles 930 have begun to accumulate near the desired treatment area 810 because of the magnetic action of magnetic source 820.

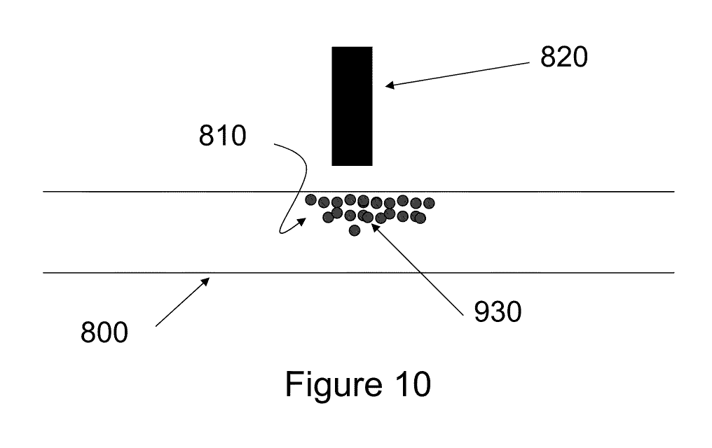

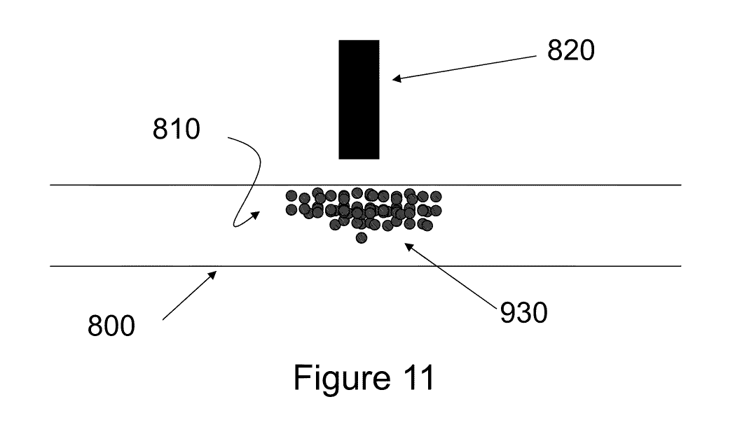

FIGS. 10 and 11 are similar to FIG. 9, except that they represent times 140 seconds and 280 seconds after introducing the magnetically sensitive particles 930, respectively. These figures show further accumulation of the particles at the desired treatment area 810.

### Therapeutic Substances

For any of the foregoing embodiments that contain or deliver drugs including from stents or from balloons such as angioplasty balloons adapted for drug delivery or drug delivery balloons can use a drug or therapeutic substance selected from those described in this section. Generally, this document uses the term “drug” and “therapeutic substance” interchangeably throughout.

Therapeutic substances are biologically active agents. Therapeutic substances can be, for example, therapeutic, prophylactic, or diagnostic agents. As used in this document, the therapeutic substance includes a bioactive moiety, derivative, or metabolite of the therapeutic substance.

Examples of suitable therapeutic and prophylactic agents include synthetic inorganic and organic compounds, proteins and peptides, polysaccharides and other sugars, lipids, and DNA and RNA nucleic acid sequences having therapeutic, prophylactic, or diagnostic activities. Nucleic acid sequences include genes, antisense molecules, which bind to complementary DNA to inhibit transcription, and ribozymes. Other examples of therapeutic substances include antibodies, receptor ligands, and enzymes, adhesion peptides, oligosaccharides, blood clotting factors, inhibitors or clot dissolving agents, such as streptokinase and tissue plasminogen activator, antigens for immunization, hormones and growth factors, oligonucleotides such as antisense oligonucleotides and ribozymes and retroviral vectors for use in gene therapy,

In other examples, the drugs or therapeutic substances inhibit vascular-smooth-muscle-cell activity. More specifically, the therapeutic substance may inhibit abnormal or inappropriate migration or proliferation of smooth muscle cells leading to restenosis inhibition. Therapeutic substances can also include any substance capable of exerting a therapeutic or prophylactic effect in the practice of the present invention. For example, the therapeutic substance could be a prohealing drug that imparts a benign neointimal response characterized by controlled proliferation of smooth muscle cells and controlled deposition of extracellular matrix with complete luminal coverage by phenotypically functional (similar to uninjured, healthy intima) and morphologically normal (similar to uninjured, healthy intima) endothelial cells.

The therapeutic substance can also fall under the genus of antineoplastic, cytostatic or anti-proliferative, anti-inflammatory, antiplatelet, anticoagulant, antifibrin, antithrombin, antimitotic, antibiotic, antiallergic and antioxidant substances.

Antineoplastic or antimitotic examples:
paclitaxel
docetaxel
methotrexate
Azathioprine
Vincristine
Vinblastine
Fluorouracil
doxorubicin hydrochloride
mitomycin

Antiplatelet, anticoagulant, antifibrin, and antithrombin examples:
Heparinoids
Hirudin
Argatroban
Forskolin
Vapiprost
Prostacyclin
prostacyclin analogues
Dextran
D-phe-pro-arg-chloromethylketone (synthetic antithrombin)
Dipyridamole
glycoprotein IIb/IIIa platelet membrane receptor antagonist antibody
recombinant hirudin and thrombin inhibitors

Cytostatic or Antiproliferative Agent Examples
Angiopeptin
angiotensin converting enzyme inhibitors
cilazapril
lisinopril
actinomycin D
dactinomycin
actinomycin IV
actinomycin I₁
actinomycin X₁
actinomycin C₁
actinomycin D derivatives or analogs

Other therapeutic substances include
calcium channel blockers
nifedipine
Colchicines
fibroblast growth factor (FGF) antagonists
omega 3-fatty acid
Fish oil
Flax seed oil
histamine antagonists
lovastatin
monoclonal antibodies (such as those specific for Platelet-Derived Growth Factor (PDGF) receptors)
Nitroprusside
phosphodiesterase inhibitors
prostaglandin inhibitors
Suramin
serotonin blockers
Steroids
thioprotease inhibitors
triazolopyrimidine (a PDGF antagonist)
nitric oxide
alpha-interferon
genetically engineered epithelial cells
antibodies such as CD-34 antibody
abciximab (REOPRO)
progenitor cell capturing antibody
pro-healing therapeutic substances (that promotes controlled proliferation of muscle cells with a normal and physiologically benign composition and synthesis product)
Enzymes
anti-inflammatory agents
Antivirals
anticancer drugs
anticoagulant agents
free radical scavengers
Estradiol
steroidal anti-inflammatory agents
non-steroidal anti-inflammatory
dexamethasone
clobetasol
aspirin
Antibiotics
nitric oxide donors
super oxide dismutases
super oxide dismutase mimics
4-amino-2,2,6,6-tetramethylpiperidine-1-oxyl (4-amino-TEMPO)
Tacrolimus
Rapamycin
rapamycin derivatives 40-O-(2-hydroxy)ethylrapamycin (everolimus)
40-O-(3-hydroxy)propyl-rapamycin
40-O-[2-(2-hydroxy)ethoxy]ethyl-rapamycin
40-O-tetrazole-rapamycin
Zotarolimus™
cytostatic agents

An example of an antiallergic agent is permirolast potassium.

The foregoing substances are listed by way of example and are not meant to be limiting. Other active agents that are currently available or that may be developed in the future are equally applicable.

Some embodiments encompass a method that comprises delivering a magnetically sensitive drug carrier near a region of the vasculature, including heart chambers and coronary arteries or other vessels, using a percutaneous delivery apparatus having a distal end and then applying magnetic energy to the vasculature using a percutaneous magnetic source apparatus, which apparatus comprises a distal end connected to a magnetic source.

Some embodiments encompass the same method or a method similar to the just-described method wherein delivery further includes placing the distal end of the percutaneous delivery apparatus near a desired treatment area of the heart chamber, coronary artery, or other vessel before delivery of the magnetically sensitive drug carrier and placing the distal end of the percutaneous magnetic source apparatus in the same or different heart chamber, coronary artery, or other vessel as the percutaneous delivery apparatus before, during, or after; before and during; before and after; during and after; or before, during, and after applying magnetic energy to the heart chamber, coronary artery, or other vessel.

Some embodiments encompass the same method or a method similar to the just-described method wherein delivery further includes placing the distal end of the percutaneous delivery apparatus near a desired treatment area of the heart chamber, coronary artery, or other vessel before delivery of the magnetically sensitive drug carrier and placing the distal end of the percutaneous magnetic source apparatus in the same heart chamber, coronary artery, or other vessel as the percutaneous delivery apparatus before, during, or after; before and during; before and after; during and after; or before, during, and after applying magnetic energy to the heart chamber, coronary artery, or other vessel.

Some embodiments encompass the same method or a method similar to a formerly described method wherein delivery further includes placing the distal end of the percutaneous delivery apparatus near a desired treatment area of the heart chamber, coronary artery, or other vessel before delivery of the magnetically sensitive drug carrier and placing the distal end of the percutaneous magnetic source apparatus in the different heart chamber, coronary artery, or other vessel as the percutaneous delivery apparatus before, during, or after; before and during; before and after; during and after; or before, during, and after applying magnetic energy to the heart chamber, coronary artery, or other vessel.

Some embodiments encompass the same method or a method similar to the just-described method wherein delivery further includes placing the distal end of the percutaneous delivery apparatus near a desired treatment area of the heart chamber, coronary artery, or other vessel before delivery of the magnetically sensitive drug carrier and placing the distal end of the percutaneous magnetic source apparatus in the same or different heart chamber, coronary artery, or other vessel as the percutaneous delivery apparatus before, during, or after; before and during; before and after; during and after; or before, during, and after applying magnetic energy with an electromagnet to the heart chamber, coronary artery, or other vessel.

Some embodiments encompass the same method or a method similar to the just-described method wherein delivery further includes placing the distal end of the percutaneous delivery apparatus near a desired treatment area of the heart chamber, coronary artery, or other vessel before delivery of the magnetically sensitive drug carrier and placing the distal end of the percutaneous magnetic source apparatus in the same or different heart chamber, coronary artery, or other vessel as the percutaneous delivery apparatus before, during, or after; before and during; before and after; during and after; or before, during, and after applying magnetic energy with a permanent magnet to the heart chamber, coronary artery, or other vessel.

Some embodiments encompass a method comprising steps of delivering a magnetically sensitive drug carrier near a region of the vasculature and applying magnetic energy to the vasculature together with imaging a vascular lesion using intravenous ultrasound before, during, or after delivering a drug carrier.

Some embodiments encompass a method comprising steps of delivering a magnetically sensitive drug carrier near a region of the vasculature and applying magnetic energy to the vasculature together with imaging a vascular lesion using intravenous ultrasound to determine a more diseased part of the lesion and applying magnetic energy to direct the magnetically sensitive drug carrier to the more diseased part of the lesion.

Some embodiments encompass a method comprising steps of delivering a magnetically sensitive drug carrier, which is a nanoparticle, microparticle, liposome, micelle, nanofiber, hydrogel, cell, or biological carrier, near a region of the vasculature and applying magnetic energy to the vasculature in such a way that the magnetic energy causes the magnetically sensitive drug carrier to localize near the region of the vasculature together with imaging a vascular lesion using intravenous ultrasound before, during, or after delivering a drug carrier.

Some embodiments encompass a method comprising steps of delivering a magnetically sensitive drug carrier, which is a nanoparticle, microparticle, liposome, micelle, nanofiber, hydrogel, cell, or biological carrier, near a region of the vasculature and applying magnetic energy to the vasculature in such a way that the magnetic energy causes the magnetically sensitive drug carrier to localize near the region of the vasculature together with imaging a vascular lesion using intravenous ultrasound to determine a more diseased part of the lesion and applying magnetic energy to direct the magnetically sensitive drug carrier to the more diseased part of the lesion.

Some embodiments encompass a method comprising steps of delivering a magnetically sensitive drug carrier, which is capable of responding to magnetic energy, which is a nanoparticle, microparticle, liposome, micelle, nanofiber, hydrogel, cell, or biological carrier and which comprises a drug, near a region of the vasculature; applying to the vasculature magnetic energy that comprises a magnetic field or a magnetic field gradient; and employing magnetic resonance imaging before, during, or after delivering the magnetically sensitive drug carrier.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true, intended, explained, disclosed, and understood scope and spirit of this invention's multitudinous embodiments and alternative descriptions.

Additionally, various embodiments have been described above. For convenience's sake, combinations of aspects composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists that specifically excludes that aspect. All patents, test procedures, and other documents cited in this specification are fully incorporated by reference to the extent that this material is consistent with this specification and for all jurisdictions in which such incorporation is permitted.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically exclude the value or the conditions for the aspect.

Finally, headings are for the convenience of the reader and do not alter the meaning or content of the disclosure or the scope of the claims.

What is claimed is:

1. A method comprising
delivering a magnetically sensitive drug carrier near a region of the vasculature and
applying magnetic energy to the vasculature.

2. The method of claim 1 wherein applying magnetic energy causes the magnetically sensitive drug carrier to localize near the region of the vasculature.

3. The method of claim 1 wherein magnetic energy comprises a magnetic field or a magnetic field gradient.

4. The method of claim 1 wherein the magnetically sensitive drug carrier is capable of responding to magnetic energy such that the magnetic energy is capable of causing a change in the motion of the magnetically sensitive drug carrier.

5. The method of claim 1 wherein the magnetically sensitive drug carrier is capable of responding to magnetic energy such that the rate that particles of the magnetically sensitive drug carrier move through the vessel that is subjected to the magnetic energy is lower than the rate that the particles move through the same or similar vessels absent magnetic energy by 10% or more; by 50% or more; by 80% or more; by 90% or more; by 95% or more; or by 99% or more.

6. The method of claim 1 wherein the magnetically sensitive drug carrier is a nanoparticle, microparticle, liposome, micelle, nanofiber, hydrogel, cell, or biological carrier.

7. The method of claim 6 wherein the magnetically sensitive drug carrier has ferromagnetic activity.

8. The method of claim 6 wherein the magnetically sensitive drug carrier is a cell or other biological carrier with an internal ferromagnetic particle.

9. The method of claim 6 wherein the magnetically sensitive drug carrier comprises ferrite particles, ferrous oxide, or rare earth particles.

10. The method of claim 6 wherein the magnetically sensitive drug carrier is attached to a moiety comprising a drug.

11. The method of claim 10 wherein the drug inhibits the migration or proliferation of smooth muscle cells.

12. The method of claim 2 wherein the magnetically sensitive drug carrier is a nanoparticle, microparticle, liposome, micelle, nanofiber, hydrogel, cell, or biological carrier.

13. The method of claim 2 wherein magnetic energy comprises a magnetic field or a magnetic field gradient
wherein the magnetically sensitive drug carrier is capable of responding to magnetic energy
wherein the magnetically sensitive drug carrier is a nanoparticle, microparticle, liposome, micelle, nanofiber, hydrogel, cell, or biological carrier and
wherein the magnetically sensitive drug carrier is attached to a moiety comprising a drug.

14. The method of claim 1 wherein applying magnetic energy to the vasculature comprises using a percutaneous magnetic source apparatus, which apparatus comprises a distal end connected to a magnetic source.

15. The method of claim 14 wherein
delivery comprises using a percutaneous delivery apparatus having a distal end and
vasculature includes heart chambers and coronary arteries or other vessels.

16. The method of claim 15 wherein delivery further comprises
placing the distal end of the percutaneous delivery apparatus near a desired treatment area of the heart chamber, coronary artery, or other vessel before delivery of the magnetically sensitive drug carrier and
placing the distal end of the percutaneous magnetic source apparatus in the same or different heart chamber, coronary artery, or other vessel as the percutaneous delivery apparatus before, during, or after; before and during; before and after;
during and after; or before, during, and after applying magnetic energy to the heart chamber, coronary artery, or other vessel.

17. The method of claim 16 wherein placing the distal end of the percutaneous magnetic source apparatus in a different heart chamber, coronary artery, or other vessel is placing such that
the distal end of the percutaneous delivery apparatus is placed into a heart chamber and the percutaneous magnetic source apparatus is placed into a different heart chamber, a different coronary artery, or a different other vessel;
the distal end of the percutaneous delivery apparatus is placed into a coronary artery and the percutaneous magnetic source apparatus is placed into a different heart chamber, a different coronary artery, or a different other vessel; or
the distal end of the percutaneous delivery source is placed into another vessel and the percutaneous magnetic source apparatus is placed into a different heart chamber, a different coronary artery, or a different other vessel.

18. The method of claim 16 wherein the distal end of the percutaneous delivery apparatus connects to an expandable member.

19. The method of claim 18 wherein the expandable member has an outer surface coated or impregnated with a magnetically sensitive drug carrier.

20. The method of claim 18 wherein the magnetically sensitive drug carrier comprises a drug.
